(19) United States
(12) Patent Application Publication
(10) Pub. No.: US 2009/0169689 A1
(43) Pub. Date: Jul. 2, 2009

(54) Evaluation Method of Partial Pressure Oxygen and Use Thereof

(75) Inventors: LOONG-SHENG CHANG, Taipei City (TW); CHING-CHANG SHIESH, Taichung County (TW); YU-TSE LIN, Kaohsiung City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 11/966,765

(22) Filed: Dec. 28, 2007

(51) Int. Cl. G01N 33/02

(52) U.S. Cl. 426/231

(57) ABSTRACT

The present invention provides a method to evaluate partial pressure oxygen based on respiration rate of horticultural product. Three zones are grouped as Crisis zone, Homeostatic zone, and Elastic stress zone. The present invention also provides a method to evaluate the most applicable low oxygen level for the storage of horticultural product.

### FIELD OF THE INVENTION

The present invention relates to a method for determining the effect of oxygen concentration on post harvest physiology of horticultural crop based on the principle of Pasteur effect.

The present invention also relates to a method for evaluating optimal oxygen concentration for horticultural crop storage.

### BACKGROUND OF THE INVENTION

The appropriate gas composition in the storage environment for horticulture crops can improve the crops' quality and life span during storage and transportation. Therefore, the approach of controlled atmosphere storage, which adjusts the gas composition in the storage environment, is an important technique on post harvest storage and transport. Most of the horticultural crops can retain better freshness and longer storage life when controlled atmosphere storage is applied.

Among various recommendations for controlled atmospheres, the combination of low oxygen and high carbon dioxide is applied widely. The combination of low oxygen and high carbon dioxide has synergistic effect on inhibiting the metabolism and ripening of horticultural crops, thus reduces the depletion of carbohydrate, organic acid and other stored nutrients of fruit as well as delay the loss of chlorophyll, the synthesis of carotenoids and anthocyanidins which cause the red and blue color on fruit, and the synthesis and oxidation of phenols which cause the brown color on fruit.

The condition of low oxygen and high carbon dioxide can also provide a stress environment for horticultural crops and may transform aerobic respiration to anaerobic respiration. Moreover, the treated horticultural crops are unable to restore regular respiration and metabolism when returning to normal atmospheric environment and causing the quality drop of horticultural crops quickly and irreversibly. Therefore, providing the appropriate condition of gas composition in accordance with the physiological characteristics of specific horticultural crops will facilitate extending their life span during storage and transportation life as well as preserving the quality of crops.

Most of the existing recommendations for controlled atmospheres have been emphasized on the approach of adjusting the ratio of oxygen and carbon dioxide simultaneously. Furthermore, when referring to oxygen, it is often limited to the investigation of oxygen concentration approaching aerobic respiration extinction point, which is the transition point of anaerobic respiration and aerobic respiration, in Pasteur effect. For easily perished fruit like tropical or subtropical fruits, this method is apparently too rough. Establish an integrated method for evaluating partial pressure oxygen to predict the physiological reaction of horticultural crops systematically and aimingly as a reflection of the quality variation of horticultural crops can significantly improve crops life span during storage and transportation as will as the quality. As a result, the export market and economic value of such horticultural crops will be expanded.

### BRIEF DESCRIPTION OF THE DRAWINGS

FIG. 1 shows the response of respiration rate of ‘Tainung No. 2’ papaya to the treatment of various oxygen partial pressures below ambient. Homeostatic zone: the range of oxygen concentration that aerobic respiration occurs. Crisis zone: the range of oxygen concentration that anaerobic respiration occurs. Elastic stress zone: the range of oxygen concentration is approximately 3˜8%.

FIG. 2 shows the variation of respiration rate and ethylene production of ‘Tainung No. 2’ papaya during partial pressure oxygen treatment. Arrows indicate the ending days (ED) of the treatment.

FIG. 3 shows the variation of L value (A), b value (B), hue angle (C), and skin color (D) of ‘Tainung No. 2’ papaya after partial pressure oxygen treatment and 8 days of shelf-life. Arrows indicate the ending days (ED) of partial pressure oxygen treatment. The average of 5 replications with standard error bar is shown. L represents the brightness of fruit and b represents the yellow color intensity of fruit.

FIG. 4 shows abnormal appearance, yellowing, and discoloration of ‘Tainung No. 2’ papaya after 1% partial pressure oxygen treatment for 8 days and 8 days of shelf-life.

FIG. 5 shows the effect of partial pressure oxygen treatment on the ethanol content of seed and pulp of ‘Tainung No. 2’ papaya.

FIG. 6 shows the variation of chlorophyll fluorescence rate of ‘Tainung No. 2’ papaya after partial pressure oxygen treatment. The average of 5 replications with standard error bar is shown.

FIG. 7 shows the evaluation of the appearance and skin gloss of ‘Tainung No. 2’ papaya after partial pressure oxygen treatment and 4 or 8 days of shelf-life.

FIG. 8 shows the appearance of ‘Tainung No. 2’ papaya after the treatment of different partial pressure oxygen and 8 days of shelf-life. Apparently, the fruit treated with oxygen concentration of 5% appears good appearance as bright yellow color, no observable rot, and high skin gloss.

FIG. 9 shows the relationship between ethanol concentration of pulp and chlorophyll fluorescence rate after 1%, 3%, 5%, 8% and 10% of partial pressure oxygen treatments.

FIG. 10 shows a flowchart of the partial pressure oxygen evaluation model.

### SUMMARY OF THE INVENTION

The present invention provides a method to evaluate partial pressure oxygen based on respiration rate of horticultural product. Three zones are grouped as-Crisis zone, Homeostatic zone, and Elastic stress zone. The present invention also provides a method to evaluate the most applicable low oxygen level for the storage and transport of horticultural product.

### DETAILED DESCRIPTION OF THE INVENTION

Low oxygen concentration is able to retard the respiration and physiological metabolism of horticultural crops such as fruit, seed, vegetable, and flower. The core element of controlled atmosphere storage technique is to evaluate appropriate low oxygen concentration. In view of the necessity of evaluating low oxygen concentration before the test of controlled atmosphere storage, the present invention provides methods to determine the endurance of horticultural product to low oxygen and the applicable range of low oxygen concentration based on the principle of Pasteur effect. A series of physiological variation of horticultural products after low oxygen treatment such as respiration rate, ethylene production, rate of color turning, days of ripening, ethanol concentration and flavor evaluation were examined thoroughly in order to establish the partial pressure oxygen evaluation model and provide references to improve controlled atmosphere storage technique. According to the present invention, the term “controlled atmosphere storage” means to reduce oxygen concentration and increase carbon dioxide concentration for the purpose of extending the storage life of horticultural products.

The term ‘Pasteur effect’ on post-harvest horticultural products mentioned in the present invention can be defined as follows: Carbon dioxide produced by respiration decreases while oxygen concentration in the atmosphere decreases. When reaching the lowest point, continuous decrease of oxygen concentration will cause rapid increase of carbon dioxide and the production of ethanol; this phenomenon is called Pasteur effect and the point is called Extinction point. The present invention is based on the concept of Pasteur effect to treat horticultural crops with different interval of oxygen concentration ranging from 1% to 20%. The respiration rate of horticultural product is then measured. In addition to Extinction point, the lowest point of respiration rate, further analysis of the respiration rate of horticultural products on different oxygen concentration is also determined. The physiological reaction can be divided into two zones, Crisis zone and Homeostatic zone, in response to oxygen concentration. In Crisis zone, horticultural crops exhibit anaerobic respiration and are unable to carry normal metabolism, resulting in the accumulation of ethanol and production of odor. In Homeostatic zone, horticultural crops exhibit aerobic respiration and normal metabolism, without the accumulation of ethanol and production of odor. Lower oxygen concentration range of the Homeostatic zone is defined as Elastic stress zone based on the physiological condition of horticultural crops, where horticultural crops exhibit lower respiration rate, delayed color-turning, and inhibition of ethylene production under normal metabolism. Finally, the quality of horticultural crops such as appearance and taste is further evaluated in Elastic stress zone to determine the most applicable low oxygen condition for longer storage life and better quality of horticultural crops.

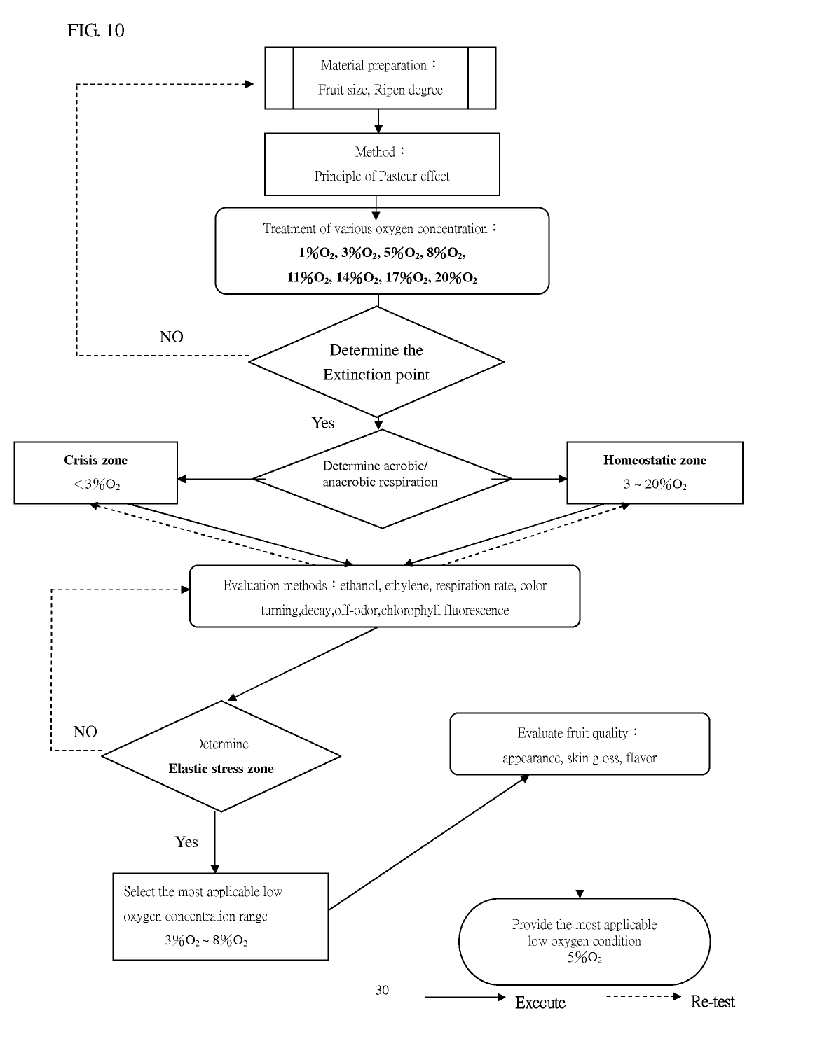

The present invention is based on the test result described above to establish a complete model for evaluating partial pressure oxygen. The scheme of the model is shown in FIG. 10. The most appropriate endurable range of low oxygen concentration for horticultural crops is first evaluated. More precise low oxygen concentration is then derived from further evaluation of the quality and physiological reaction of horticultural crops in this range. The precise low oxygen concentration provides longer storage life and better quality of horticultural crops.

Accordingly, the present invention provides a method for determining the Extinction point, comprising the following processes: (a) treating a horticultural crop with different interval of oxygen concentration in the range of 1%˜20% O₂ level and (b) measuring the respiration rate of horticultural crops to get the Extinction point which is the lowest point of respiration rate. The horticultural crop mentioned in the present invention comprises but not limit to is fruit such as papaya, seed, vegetable or flower.

In one preferred embodiment of the present invention, the Extinction point of papaya is at 3% of oxygen concentration.

In another preferred embodiment of the present invention, Crisis zone, Homeostatic zone and Elastic stress zone of the horticultural crop are further determined. Crisis zone is the range of oxygen concentration in which the horticultural crop exhibits anaerobic respiration and is unable to carry normal metabolism, resulting in the accumulation of ethanol and production of off-odor. The Crisis zone of papaya is below 3% of oxygen concentration. Homeostatic zone is the range of oxygen concentration in which the horticultural crop exhibits aerobic respiration and normal metabolism, without the accumulation of ethanol and production of off-odor. The Homeostatic zone of papaya is at 3%˜20% of oxygen concentration. Elastic stress zone is the low oxygen range of Homeostatic zone; in which, the horticultural crop exhibits lower respiration rate, delayed color-turning, and inhibition of ethylene production under normal metabolism. The Elastic stress zone of papaya is at 3%˜8% of oxygen concentration.

In still another preferred embodiment of the present invention, the quality of horticultural crops in Elastic stress zone is further evaluated. The most applicable low oxygen condition provides longer storage life and better quality of the horticultural crop. The most applicable low oxygen condition of papaya is at 5% of oxygen concentration.

The present invention also provides a method for evaluating the most applicable oxygen concentration for the storage of horticultural crops, comprising (a) the method for determining Extinction point described above and (b) evaluation of physiological condition or quality of horticultural crops. The physiological condition of horticultural crops comprises, but not limit to, ethanol concentration, ethylene production rate, respiration rate, rate of color turning, rate of decay, the intensity of off-odor or chlorophyll fluorescence rate. The quality of horticultural crops comprises, but not limit to, appearance, taste or flavor.

### EXAMPLE

Papaya was taken as an example in the present invention, because it is one of the most unendurable horticultural crops for storage. As shown in the present invention, the successful application of this method for papaya storage promises its usage for other horticultural crops. Examples below are non-limiting and are merely representative of various aspects and features of the present invention.

### Example 1

### Materials

The ‘Tainung No. 2’ papaya of the present invention was grown in net house. Each of the post-harvest fruit was wrapped in a ‘Shu-guo’ bag and loaded in basket vertically. Totally three batches of fruit were harvested for replication. The maturity of harvested fruit was 15˜25% of yellow coloration. The average weight of tested fruit was 809.3±14.4 g. Fruit met the criteria for testing were selected randomly, and soaked in 1000×TBZ for 3 minutes to avoid the interference of fungi. After soaking in TBZ and the following 2 hours of air dry, the test began immediately. When half of the total treated fruit reached 75% of ripening, the test was stopped.

### Example 2

### Tolerance of Fruit to Low Oxygen Concentration

There were total three replications of the test. In the first two replications, the fruits were placed in acrylics respiration vats with the capacity of 7 liters, and the oxygen concentration was set to be 1%, 3%, 5%, 8%, 10%, 11% and 20% for the treatment. In the third replication, the fruits were placed in acrylics respiration vats with the capacity of 62 liters, and the oxygen concentration was set to be 1%, 3%, 5%, 8%, 11%, 14%, 17% and 20% for the treatment. The flow rate of gas was controlled by water level and the capillary on the flow regulator. The gas flowed through the respiration vat came from outdoor fresh air; after mixing with nitrogen of different flow rate to make up various oxygen concentration, it was introduced into the vat with constant temperature of 25° C. The oxygen concentration was measured at the same time everyday by collecting 10 ml of gas sample with 30 ml syringe and analyzing the gas sample with Oxygen analyzer (Toray mocon). The value was read immediately after injecting the gas sample into the analyzer and presented as percentage (%). To measure the respiration rate, 1 ml of gas sample was collected by a syringe, and the carbon dioxide concentration of the sample was analyzed by IR-analyzer (Maihak, UNOR610) for the calculation of respiration rate. The unit of respiration rate was represented as “ml CO₂/kg-hr” and the calculating formula was as follows:

$$\frac{Peak of sample - Peak of air}{Peak of standard} * concentration of standard ( \% ) * 10 * flow rate ( L \text{/} hr ) / fresh weight of fruit ( Kg )$$

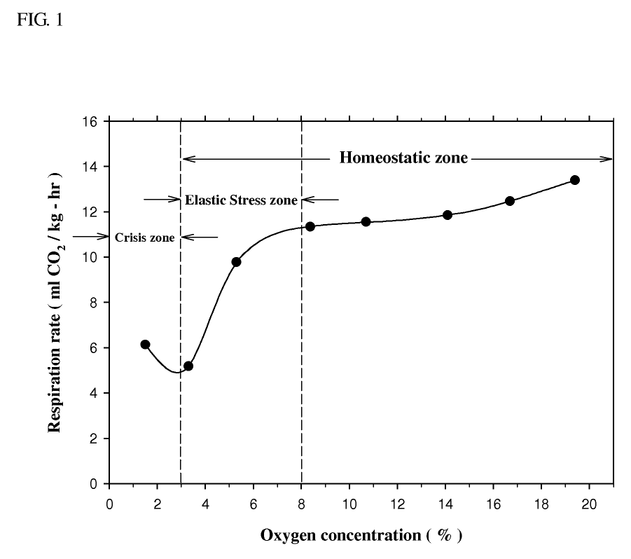

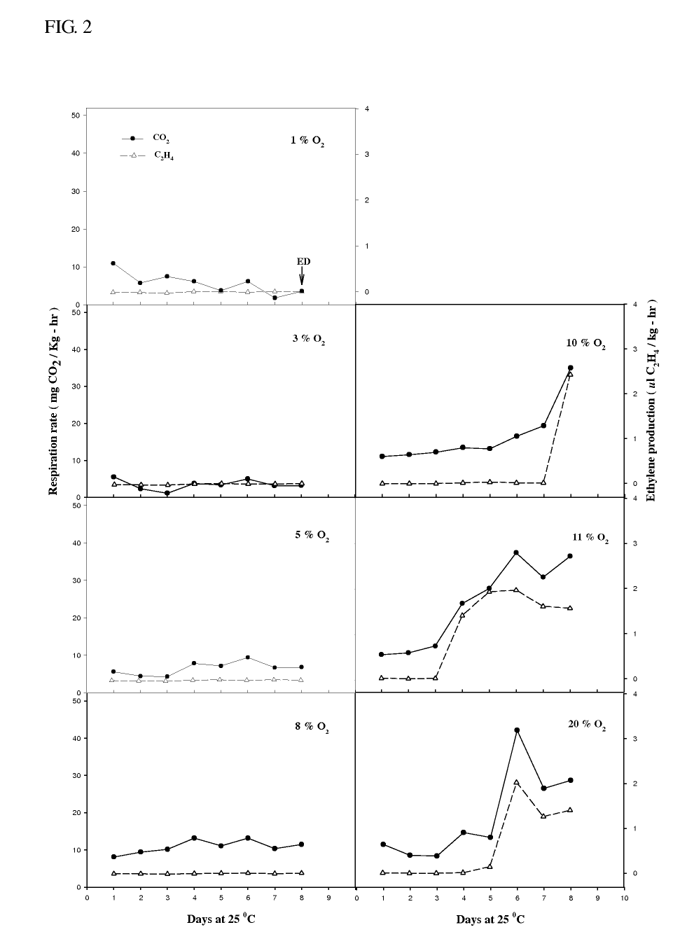

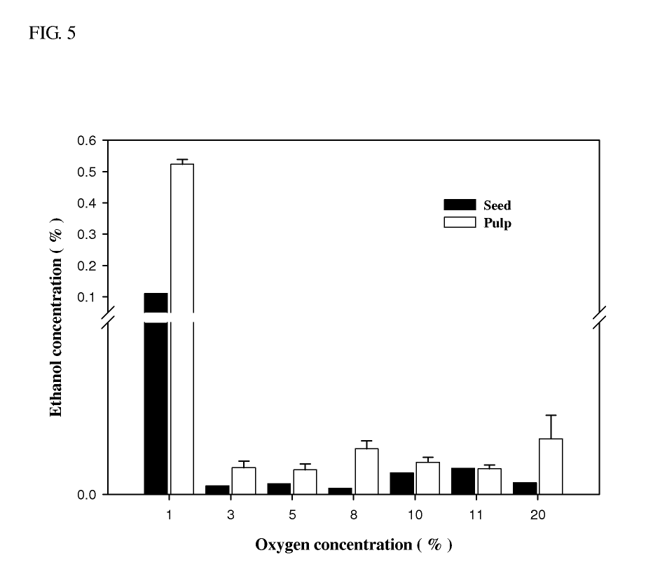

The result showed that 3% of oxygen concentration was the Extinction point (FIG. 1). Carbon dioxide concentration increased when oxygen concentration is higher than 3% or lower than 3%. The hindrance of aerobic respiration accompanied with ethanol production (FIG. 5), off-odor of fruit chamber (Table 2), and increased respiration rate (FIG. 2) appeared when the oxygen concentration was lower than 3%.

### Example 3

### Effect of Oxygen Concentration on the Respiration Rate and Ethylene Production of Fruit

The respiration rate was measured and calculated as example 2. The method for measuring ethylene production was as follows: collecting the gas sample with 1 ml syringe, analyzing the concentration of ethylene by Gas chromatograph (Shimadzu, Model GC-8A-FID), and calculating the ethylene production by the following formula

$$\frac{( Peak of sample * attenuation ratio ) - ( Peak of air * attenuation ratio )}{( Peak of standard * attenuation ratio )} * concentration of standard ( ppm )$$

*flow rate(L/hr)/fresh weight of fruit(Kg).

The unit of the Ethylene production of fruit is “μl C₂H₄/kg-hr”.

When treated with 1˜8% oxygen concentration, the ethylene production of fruit was lower than 0.02 μl C₂H₄/Kg-hr (FIG. 2). The respiration rate was affected as well as shown in FIG. 2.

### Example 4

### Effect of Oxygen Concentration on Appearance Performance of Fruit

Two points on the equator of fruit were selected (one was the middle point of the green region and the other was the point 180⁰ opposite to it) for each of the tested fruit; L value, b value and Hue angle of these two points were then measured by Handy colormeter (Nippon Denshoku, Model NR-3000). “L value” indicated the brightness of fruit, and the higher of the value meant the brighter of the fruit. “b value” indicated the yellow color intensity of fruit, and the higher of the value meant the more intense of yellow color. Hue angle was measured as h⁰=tan⁻¹ b*/a*; 0⁰ indicated red-purple color, 90⁰ indicated yellow color, 180⁰ indicated blue-green color, and 270⁰ indicated blue color. Besides, the “Skin color” was observed by naked eyes; 0% indicated green fruit while 25%, 50%, 75% and 100% indicated 25%, 50%, 75% and 100% of yellow coloration respectively.

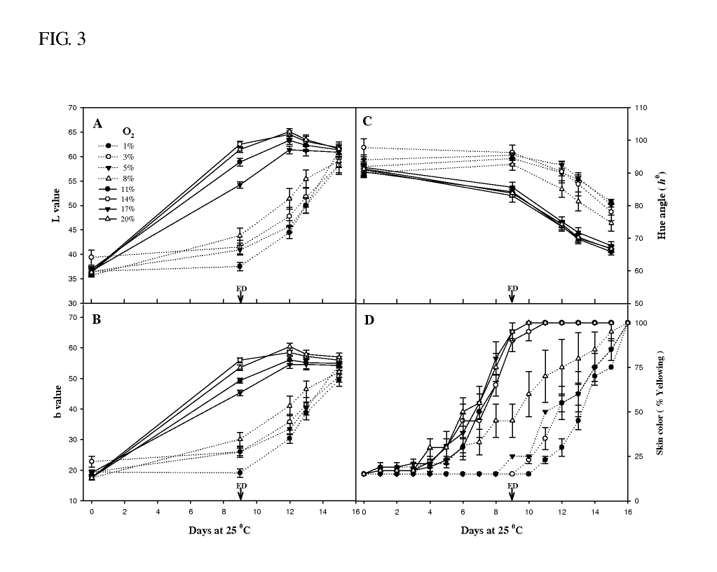

From the observation of yellow coloration, the fruit treated with 1˜8% oxygen concentration at 25° C. completely turned yellow at the 16^{th} day of ripening while the fruit treated with 11˜20% oxygen concentration completely turned yellow at the 10^{th} and 11^{th} day of ripening (FIG. 3D, Table 1). At the 8^{th} day of treatment, the fruit treated with 11˜20% oxygen concentration could be seen to reach the yellow coloration of 75% while the fruit treated with 1˜8% oxygen concentration reached the yellow coloration of 15˜50%. It revealed that low oxygen concentration could delay the rate of peel color turning.

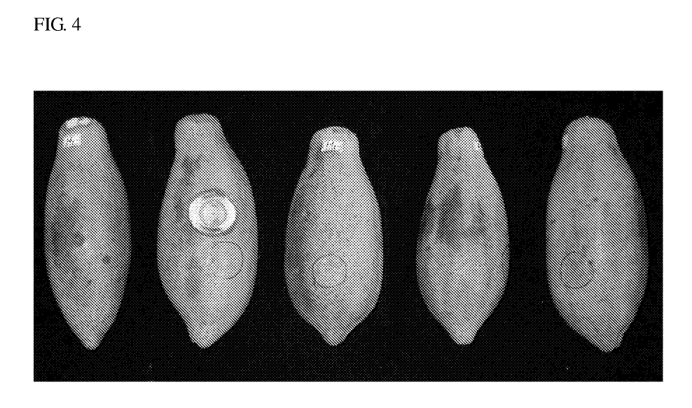

On the other hand, L value (FIG. 3A), b value (FIG. 3B) and Hue angle (FIG. 3C) had the same tendency and condition of color turning at the 9^{th} day of treatment and thus the two oxygen concentration regions, 1˜8% and 11˜20%, could easily be distinguished as two groups in the figures. The treatment of 1˜8% oxygen concentration affected the L value and b value and thus showed delay of fruit color turning rate, low skin gloss and dark green appearance. The L value and b value of the fruit treated with low oxygen concentration of 1% were significantly different to the values of the fruit treated with 3˜20% of oxygen concentration. The treatment of 1% oxygen concentration caused unhealthy appearance, uneven color turning and discoloration to fruit (FIG. 4).

TABLE 1

| | | |
|---|---|---|
| O₂ | | |
| concentration | Time to 100% yellowing^{y} | Decay^{z} |

| (%) | (days) | 4 D^{x} | 8 D |
|---|---|---|---|
| 1% | 16 | 1 | 2 |
| 3% | 16 | 1 | 1 |
| 5% | 16 | 0 | 0 |
| 8% | 16 | 1 | 4 |
| 11% | 11 | 5 | 5 |
| 14% | 11 | 5 | 5 |
| 17% | 10 | 3 | 5 |
| 20% | 10 | 4 | 5 |

^{z}Decay evaluated in fruit ripening of 4 and 8 days of shelf-life after pO₂ treatment at 25° C. Decay was considered as the fruits required to reach 10% of surface decay at 25° C. Each value is counted decay fruits number of 5 fruits in sampling days.
^{y}100% Yellowing evaluated after ripening days at 25° C. Each value is counted 100% Yellowing fruits number of 5 fruits.
^{x}Sample days of shelf-life at 25° C.

### Example 5

### Effect of Partial Pressure Oxygen Treatment on Ethanol Concentration of Seed and Pulp in Fruit Chamber

When half of the treated fruit reached 75% of ripening (totally 8 days), the test was stopped and all of the samples were taken out for the following analysis of ethanol concentration.

(1) Pulp tissue: 1 g of pulp was taken from the equator of fruit and quick-frozen by liquid nitrogen. The frozen pulp was then put into a 12 ml test tube and stoppered using rubber bung before stored in a −5° C. refrigerator. For analysis, the pulp was first thawed at 1° C. for 1 hour, and then shaken in a 30° C. incubator for 1 hour. The gas on the upper level of the test tube was collected by a 1 ml syringe and injected into Gas chromatograph (Schimadzu, Model GC-8A-FID) to analyze the ethanol concentration. There were 2 replications for each fruit.
(2) Seed: 10 g of seeds was taken out immediately after cutting open the equator of the fruit; it was put into an 50 ml Erlenmeyer flask and stoppered using rubber bung for 1 hour before shaking in a 30° C. incubator for 1 hour. The gas on the upper level of the flask was collected by a 1 ml syringe and injected into Gas chromatograph (Schimadzu, Model GC-8A-FID) to analyze the ethanol concentration. There was no replication.

While the oxygen concentration was lower than 3%, anaerobic respiration of fruits induced fermentation resulting in ethanol accumulation. After the treatment of partial pressure oxygen with oxygen concentration of 1%, 3%, 5%, 8%, 10%, 11% and 20% respectively, the ethanol concentration of seed and pulp in fruit chamber was analyzed. After the treatment of 1% oxygen concentration, the ethanol concentration of seed was 0.11% and the ethanol concentration of pulp was 0.52%±0.02. After the treatment of 3˜20% oxygen concentration, the ethanol concentration of seed was 0.00087˜0.005% and the ethanol concentration of pulp was 0.0036˜0.0096% with significantly difference from that of 1% oxygen concentration treatment (FIG. 5).

### Example 6

Changes of Chlorophyll Fluorescence of Fruit after the Treatment of Different Oxygen Concentration

At room temperature of 25° C., the chlorophyll fluorescence of fruit treated with two groups of oxygen concentrations was measured before and after the treatment. One group was treated with oxygen concentration of 1%, 3%, 5%, 8%, 11%, 14%, 17% and 20%, the other group was treated with oxygen concentration of 1%, 3%, 5%, 8%, 10%, 11% and 20%. Two observation values were taken for each fruit; there were 5 replications for the first oxygen concentration group and 2 replications for the second oxygen concentration group. After being taken out from the respiration vats, the fruits were covered with black flannelette for 30 minutes for dark adaptation. The chlorophyll fluorescence was measured immediately after the process of dark adaptation by connecting Portable Chlorophyll Fluorometer (MiNi-PAM, Walz Germany) with Distance leaf-clip and illuminating the skin of fruit using a 0.8 s saturating pulse of 10000 μmol m⁻² s⁻¹ PAR. The positions of measurement were the green spot selected from the ‘Fix-circle’ of fruit and the point 180⁰ opposite to the green spot; these two points were measured for each fruit. The measurement was indicated as Fv/Fm value. Fo (minimal fluorescence) indicated the fluorescence of dark-treated materials with PS II reaction center at the state of full opening. Fm (maximal fluorescence) indicated the fluorescence of dark-treated materials with PS II reaction center at the state of full closed after illuminating with saturating pulse. Fv (variable fluorescence) was the difference of Fm−Fo. The efficiency of photosynthesis of PS II was usually measured as the quotient of Fv/Fm. The Red LEDs (light emitting diodes) of MiNi-PAM was adjusted to be 60% of maximum light intensity; the wavelength of the light source was 650 nm; the time of recording was set to be 1 second.

Fruits were treated with various concentration of oxygen at 1%, 3%, 5%, 8%, 11%, 14%, 17% and 20% under 25° C. The experiment stopped when half of the treated fruit reached 75% ripening (total 8 days). Samples were taken out for dark adapt and analyzed by portable chlorophyll fluorometer.

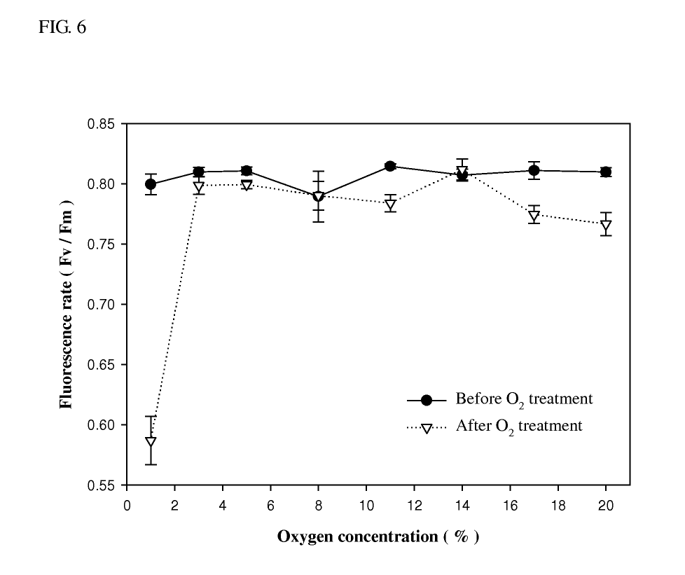

The Fv/Fm value of fruit treated with 1% of oxygen concentration was 0.59±0.02 which was significantly different to the other oxygen concentration treatments (FIG. 6).

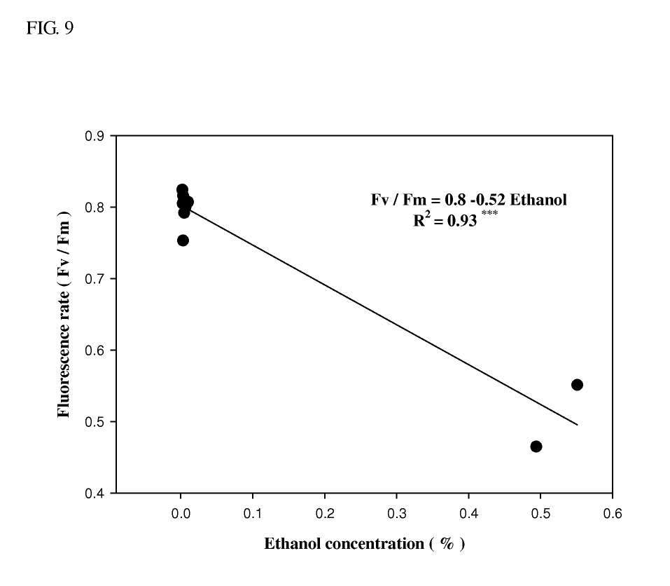

As for the relationship between chlorophyll fluorescence rate (Fv/Fm) and ethanol concentration of pulp, the fruit treated with the oxygen concentration of 1% 3%-5%-8% and 10% were selected from the group of fruit treated with the oxygen concentration of 1%-3%-5%-8%-10%-11% and 20% and tested. The result showed that the value of Fv/Fm was negatively correlated with ethanol concentration of pulp (r=−0.96, P<0.0001) (FIG. 9).

### Example 7

Effect of Oxygen Concentration on Total Soluble Solids, Firmness and Off-Odor of Fruit

After crosscutting fruit at its equator and squeezing juice from pulp, the Total soluble solid (TSS) was measured by Hand refractometer (Atago, Model N1). The unit was indicated as ⁰Brix.

As for the firmness of fruit, the maximum weight per unit area to puncture the pulp was measured by Penetromenter F327 at the section of pulp 0.2 cm away from the chamber of fruit. Totally 4 points were measured for each fruit for the average. The unit shown on the meter was 1 b/cm² which was converted to N (Newton) as the unit to indicate firmness.

The firmness of fruit treated with the oxygen concentration of 1% was 24.1 N, which was significantly different to the fruit treated with other oxygen concentration. There was no significant difference of Total soluble solids between fruit treated with various oxygen concentrations. The fruit treated with the oxygen concentration of 1% produced off-odor (Table 2).

TABLE 2

| O₂ concentration | | TSS | |
|---|---|---|---|
| (%) | Firmness (N)^{z} | (⁰Brix) | Off-odor^{y} |
| 1% | 24.1 | 10.2 | 5 |
| 3% | 11.4 | 10.9 | 0 |
| 5% | 14.0 | 11.1 | 0 |
| 8% | 10.8 | 11.0 | 0 |
| 11% | 9.1 | 10.6 | 0 |
| 14% | 9.1 | 10.1 | 0 |
| 17% | 9.7 | 11.2 | 0 |
| 20% | 9.1 | 9.9 | 0 |
| LSD_{0.05}^{x} | 3.4 | 0.9 | — |

^{z}Fruit evaluated after pO₂ treatment and 8 days of shelf-life at 25° C. Each value is a mean of 5 replicates.
^{y}Off-odor was considered as the fruits smell of odor as ethanol. Each value is counted off-odor fruits number of 5 fruits.
^{x}Mean separation within columns by Least significant difference, 5% level.

### Example 8

### Effect of Oxygen Concentration on Fruit Decay

Rate of decay was determined by observing and recording the percentage of moldy and rotten area relative to the total surface area of fruit. The number of fruit and days to reach 10% of surface decay is recorded.

The number of decay fruit reached 10% of surface decay at the fourth and eighth day of shelf-life after the treatment of 1%, 3%, 5%, 8%, 11%, 14%, 17% and 20% oxygen concentration at 25° C. for eight days was evaluated. At the fourth day of shelf-life after various oxygen concentration treatment, the decay rate of fruit treated with 1˜8% oxygen concentration was significantly different to that treated with 11˜20% oxygen concentration (Table 1). At the eighth day of shelf-life, the fruit treated with 5% of oxygen concentration exhibited good appearance without decay, and the decay rate is below 3%.

### Example 9

### Effect of Oxygen Concentration on Appearance, Skin Gloss and Flavor

The appearance and skin gloss of fruit were determined by one person. The fruits were treated with various oxygen concentrations at 25° C. until half of the treated fruits reached 75% of ripening. The fruits were then stored on shelf at 25C. The fruits were evaluated twice when half (3 to 4 days of fruit natural ripening) and all treated fruits reached 90˜100% of ripening. The appearance was evaluated by observing the diseased or rotten appearance on the surface of fruit and the integral acceptance of the fruits. Six ranks were listed as follows: 0=very poor, 1=poor, 2=fair, 3=good, 4=very good, and 5=excellent. The skin gloss was also ranked into six grades: 0=normal gloss, 1, 2, 3, 4, and 5=high gloss.

When all of the fruit reached 90˜100% of ripening, the sensory evaluation was also determined. The fruits were crosscut into pieces (about 5×5×1 cm³ for each piece) and two replications were performed. The Total soluble solids of the fruits was about 10.2˜10.6 ⁰Brix. Eight panelists were invited to taste and evaluate the samples. There were three ranks for the score of flavor: 1=poor, 2=fair, and 3=good.

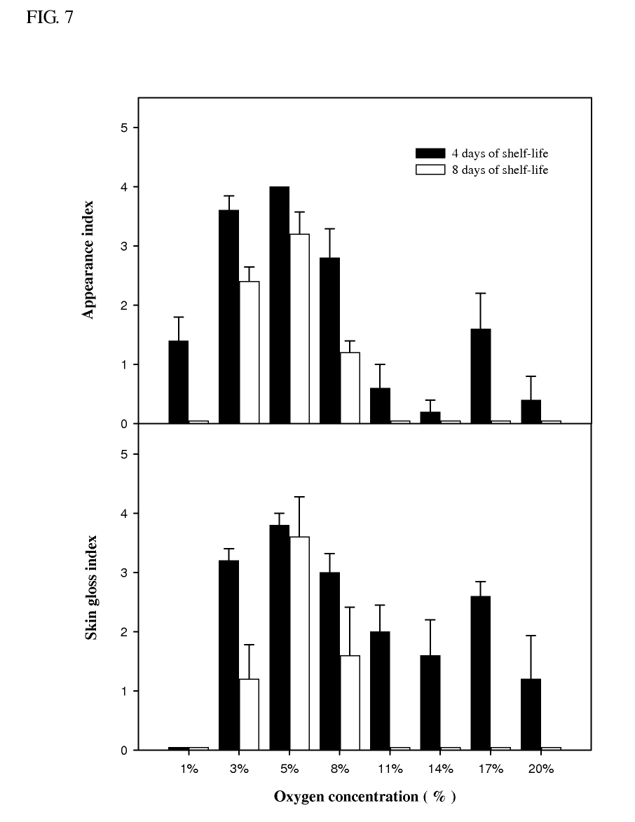

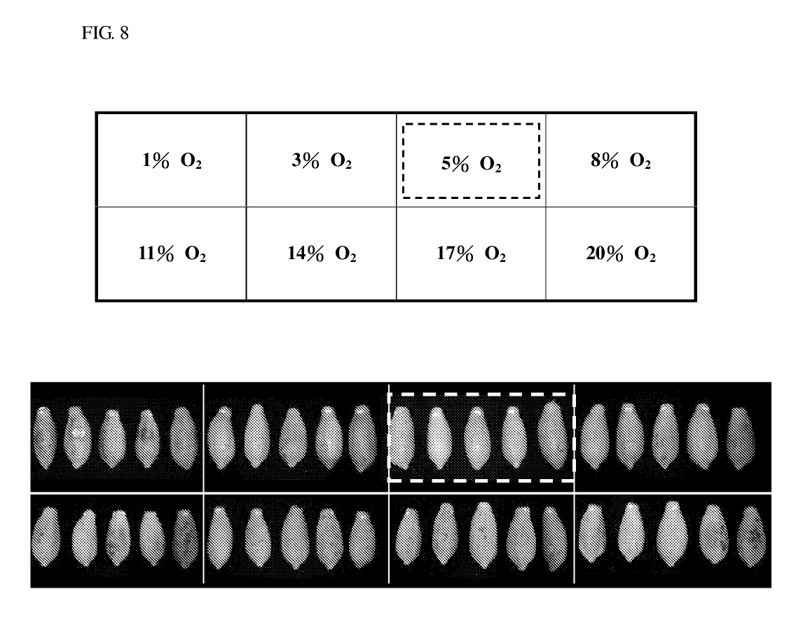

The appearance and sensory evaluation of fruits treated with oxygen concentration of 1%, 3%, 5%, 8%, 11%, 14%, 17% and 20% were shown in FIG. 7 and Table 3, respectively. The fruit treated with oxygen concentration of 5% showed good appearance with bright yellow color, no observable rot, and high skin gloss (FIG. 8 and Table 3). The result of sensory evaluation at the eighth day of storage showed that the fruit treated with oxygen concentration of 14% had worse flavor.

TABLE 3

| O₂ concentration | | | | |
|---|---|---|---|---|
| (%) | Appearance^{z} | Flavor | Skin gloss | Weighted score^{y} |
| 1% | 0.0 | 1.4 | 0.0 | 0.5 |
| 3% | 2.4 | 2.1 | 1.2 | 2.1 |
| 5% | 3.2 | 1.8 | 3.6 | 2.8 |
| 8% | 1.2 | 1.8 | 1.6 | 1.5 |
| 11% | 0.0 | 1.9 | 0.0 | 0.6 |
| 14% | 0.0 | 1.0 | 0.0 | 0.3 |
| 17% | 0.0 | 2.4 | 0.0 | 0.8 |
| 20% | 0.0 | 2.0 | 0.0 | 0.7 |
| Pr > Chi-Square^{x} | <0.0001 | <0.0021 | <0.0002 | — |

^{z}Quality of fruit evaluated in fruit ripening of 8 days of shelf-life after pO₂ treatment at 25° C. Appearance and skin gloss value is a mean of 5 replicates. Flavor value is a mean of 8 replicates.
^{y}Weighted score is sum of appearance, flavor and skin gloss score which multiplied by weight value of 3, 2 and 1, respectively and divided by sum of weight value.
^{x}Pr > Chi-Square separation within rows by the NPAR1WAY procedure and Kruskal-Wallis test, 5% level.

While the invention has been described and exemplified in sufficient detail for those skilled in this art to make and use it, various alternatives, modifications, and improvements should be apparent without departing from the spirit and scope of the invention.

What is claimed is:

1. A method for determining the Extinction point, comprising the following processes: (a) treating a horticultural crop with different interval of oxygen concentration in the range of 1%˜20% O₂ level and (b) measuring the respiration rate of horticultural crops to get the Extinction point which is the lowest point of respiration rate.

2. The method of claim 1 wherein said horticultural crop is fruit, seed, vegetable or flower.

3. The method of claim 2 wherein said fruit is papaya.

4. The method of claim 1 which further determines Crisis zone, Homeostatic zone and Elastic stress zone of the horticultural crop.

5. The method of claim 3 wherein the Extinction point of papaya is at 3% oxygen concentration.

6. The method of claim 4 wherein said Crisis zone is a range of oxygen concentration in which the horticultural crop exhibits anaerobic respiration and is unable to carry normal metabolism, resulting in the accumulation of ethanol and production of off-odor.

7. The method of claim 17 wherein said Crisis zone of papaya is below 3% of oxygen concentration.

8. The method of claim 4 wherein said Homeostatic zone is a range of oxygen concentration in which the horticultural crop exhibits aerobic respiration and normal metabolism, without the accumulation of ethanol and production of off-odor.

9. The method of claim 18 wherein the Homeostatic zone of papaya is at 3%˜20% of oxygen concentration.

10. The method of claim 4 wherein said Elastic stress zone is at the low oxygen concentration range of Homeostatic zone; in which, the horticultural crop exhibits lower respiration rate, delayed color-turning, and inhibition of ethylene production under normal metabolism.

11. The method of claim 19 wherein said Elastic stress zone of papaya is at 3%˜8% of oxygen concentration.

12. The method of claim 4 which further determines the most applicable low oxygen condition in the Elastic stress zone, in which the storage life of the horticultural crop is longer while the quality of the horticultural crop is maintained high.

13. The method of claim 20 wherein the most applicable low oxygen condition of papaya is at 5% of oxygen concentration.

14. A method for evaluating the most applicable oxygen concentration for the storage of horticultural crops, comprising (a) the method of claim 1 and (b) evaluating physiological condition or quality of horticultural crops.

15. The method of claim 14 wherein the physiological condition of horticultural crops comprises ethanol concentration, ethylene production rate, respiration rate, rate of color turning, rate of decay, the intensity of off-odor or chlorophyll fluorescence rate.

16. The method of claim 14 wherein said quality of horticultural crops comprises appearance, taste or flavor.

17. The method of claim 6, wherein the horticultural crop is papaya.

18. The method of claim 8, wherein the horticultural crop is papaya.

19. The method of claim 10, wherein the horticultural crop is papaya.

20. The method of claim 12, wherein the horticultural crop is papaya.
